(19) United States
(12) Patent Application Publication
(10) Pub. No.: US 2025/0213185 A1
(43) Pub. Date: Jul. 3, 2025

(54) DETERMINING A PATIENT’S RESPONSE TO A TREATMENT IN MULTIPLE MYELOMA

(71) Applicants: Hoffmann-La Roche Inc., Little Falls, NJ (US); Genentech, Inc., San Francisco, CA (US)

(72) Inventors: Shaji KUMAR, Rochester, MN (US); Stefka Grigorova TYANOVA, Basel (CH); Mellissa WILLIAMSON, San Francisco, CA (US); Tao XU, Basel (CH)

(73) Assignees: Hoffmann-La Roche Inc., Little Falls, NJ (US); Genentech, Inc., San Francisco, CA (US)

(21) Appl. No.: 18/846,553

(22) PCT Filed: Mar. 13, 2023
(86) PCT No.: PCT/EP2023/056385

§ 371 Date: Sep. 12, 2024

(30) Foreign Application Priority Data
Mar. 14, 2022 (EP) 22161961.2

(51) Int. Cl. A61B 5/00; G16H 10/20; G16H 50/30

(52) U.S. Cl. CPC A61B 5/4848; G16H 10/20; G16H 50/30

(57) ABSTRACT

The present disclosure relates to a computer-implemented method of a computer-implemented method of determining a patient's response to a treatment in multiple myeloma. The method comprises:
providing results of a series of predefined consecutive tests on the patient,
determining a response at time t as a function of a test result of the time t and a subsequent test result of a time t+1.

### TECHNICAL FIELD

The present application generally relates to the field of determining a patient's response to a cancer treatment, more in particular in multiple myeloma, using a computer-implemented method.

### BACKGROUND

Multiple myeloma (MM) is a cancer of the bone marrow that results in production of abnormal blood plasma cells. Plasma cells are a type of white blood cell that normally produces antibodies.

Different types of treatments for multiple myeloma are known, e.g. targeted therapies, chemotherapy, stem cell transplant and other measures. The treatment is focused on decreasing the clonal plasma cell population and consequently decrease the symptoms of disease. The present disclosure is not limited to any specific type of treatment.

Conventionally, a response to treatment in multiple myeloma may be assessed using the International Myeloma Working Group (IMWG) response criteria based on MM-specific laboratory measurements. Such measurements comprise e.g.: bone marrow biopsies of the patient, a measurement of the monoclonal Myeloma (M) protein in the patient's serum, i.e. the serum M-protein, a measurement of the (M) protein in the patient's urine, i.e. the urine M-protein, or a measurement of the patient's free light chain (FLC) absolute difference or FLC ratio, i.e. the FLC absolute difference FLC ratio, cf.: Kumar S et al. International Myeloma Working Group consensus criteria for response and minimal residual disease assessment in multiple myeloma. Lancet Oncol. 2016 August; 17(8): e328-e346. doi: 10.1016/S1470-2045(16)30206-6. PMID: 27511158.

However, healthcare providers do not routinely collect all measurements (e.g., bone marrow biopsies) required by IMWG response criteria, so electronic health records (EHRs) are often incomplete to assess a response to a MM treatment.

Accordingly, in the absence of recorded measurement data in patients' EHRs, there is a need to develop a method of determining a patient's response to a treatment in multiple myeloma that do not require all measurements of the IMWG criteria but merely available, routinely collected data for MM patients. At the same time, it is desirable that the reliability of the determined response is not decreased.

### SUMMARY

A simplified summary of some embodiments of the disclosure are provided in the following to give a basic understanding of these embodiments and their advantages. Further embodiments and technical details are described in the detailed description presented below.

According to an embodiment, a computer-implemented method of determining a patient's response to a treatment in multiple myeloma comprises: providing results of a series of predefined consecutive tests on the patient; determining a response at time t as a function of a test result of the time t and a subsequent test result of a time t+1.

In other words, the results of at least two consecutive tests at times t, t+1 (and optionally further times t+n and/or t-n) may be combined to determine the response at time t.

Hence, by taking into account two consecutive tests t, t+1, the result of the subsequent test at time t+1 may be used to verify the result of the test at time t. For example, if the result of the subsequent test at time t+1 confirms the result of the test at time t, the test result at time t may be considered as valid and may be used to determine the response.

By providing such a method, it becomes possible to increase the reliability of the determined patient's response. Therefore, the absence of measurements defined in the IMWG criteria can be compensated.

Each test may comprise at least one predefined type of quantitative measurement of the patient or a combination of at least two predefined types of quantitative measurements of the patient.

Accordingly, different measurements may be combined to determine a response.

The response may be a qualitative response value and/or the response may comprise a plurality of increasing (e.g. qualitative) response levels. It is however noted in this context that the term “qualitative” shall merely express that the response determined by the computer-implemented method corresponds to a (conventional) qualitative evaluation of the patient's health made by a physician according to the IMWG criteria. The determined responses may anyway be based on quantitative measurements, so the response may also be referred to as a quantitative determination.

The plurality of increasing (e.g. qualitative) response levels may comprise:
a partial response (PR)
a very good partial response (VGPR),
a complete response (CR), and
a stringent complete response (SCR).

The response and/or the response level at time t may be determined as a function of the test result of time t (and optionally of the test result of time t+1) using a predefined algorithm.

In particular, the subsequent test result of time t+1 may be used to confirm the determined response and/or determined response level at time t.

Furthermore, in case the subsequent test result of time t+1 is worse than the test result of time t, the determined response level at time t may be decreased as a function of the subsequent test result and/or is decreased to a response level corresponding to the subsequent test result.

Accordingly, if the result of the subsequent test at time t+1 disagrees with the result of the test at time t (i.e. is worse than the test result of time t), the determined test result at time t may be amended or invalidated as a function of the result of the test at time t using the predefined algorithm.

The different types of measurements may comprise at least one of:
measurement of the (e.g. monoclonal) myeloma (M) protein in the patient's serum, i.e. the serum M-protein,
measurement of the (e.g. monoclonal) M-protein in the patient's urine, i.e. the urine M-protein,
measurement of the patient's free light chain (FLC) absolute difference, i.e. the FLC absolute difference, and
measurement of the patient's free light chain (FLC) ratio, i.e. the FLC ratio.

The FLC absolute difference may be defined as |FLC lambda−FLC kappa|. The FLC ratio may be defined as (FLC kappa/FLC lambda).

It is desirable that the tests exclude a bone marrow biopsy of the patient. In other words, the response may be determined without (or in absence of) test results of a bone marrow biopsy of the patient.

A partial response (PR) and/or a very good partial response (VGPR) may be determined as a function of the measured serum M-protein or the measured urine M-protein, and optionally of the FLC absolute difference. Said rule may be comprised by the predefined algorithm. Accordingly, the predefined algorithm may generally define, in which cases a PR or VGPR is determined.

A partial response (PR) may be determined as a function of: a reduction of more than 50% of serum M-protein, or a reduction of urine M-protein by more than 90% in 24 hours or to less than 200 mg in 24 hours, or, if the serum and urine M-protein are unmeasurable, a more than 50% decrease in the difference between involved and uninvolved FLC levels.

Said rule may be comprised by the predefined algorithm. Accordingly, the predefined algorithm may define, in which cases a PR is determined.

As mentioned, there may be two types of FLCs, kappa and lambda. Generally, the one having a higher value may be considered as the involved FLC. For example, if Kappa/Lambda ratios is above e.g. 1.65, the Kappa level may be the involved FLC level. Otherwise, if the ratio is below e.g. 0.26, then Lambda level may be the involved FLC level.

Involved free light chains may be abnormally high and associated with M protein. Uninvolved free light chains may be normal levels not associated with M protein.

A very good partial response (VGPR) is determined as a function of:
a detectability of serum and urine M-protein by immunofixation but not on electrophoresis, or
a reduction of more than 90% in serum M-protein in combination with urine M-protein level being less than 100 mg in 24 hours.

Said rule may be comprised by the predefined algorithm. Accordingly, the predefined algorithm may define, in which cases a VGPR is determined.

A complete response (CR) and/or stringent complete response (SCR) may be determined as a function of the measured serum M protein being less than 1 g/dL and the measured urine M protein being less than 200 mg during 24 hours.

Said rule may be comprised by the predefined algorithm. Accordingly, the predefined algorithm may generally define, in which cases a CR and/or a SCR is determined.

A complete response (CR) may be determined as a function of: a negative immunofixation on the serum and urine.

Said rule may be comprised by the predefined algorithm. Accordingly, the predefined algorithm may define, in which cases a CR is determined.

A stringent complete response (SCR) may be determined as a function of: a negative immunofixation on the serum and urine, and an FLC ratio being in a predefined normal range.

Said rule may be comprised by the predefined algorithm. Accordingly, the predefined algorithm may define, in which cases a SCR is determined.

At least one of the times t and t+1, or each of the times t, t+1, t+n (and/or t-n) of the series may define a time range or time period, e.g. of a predefined length. Accordingly, in the present disclosure a time t, t+1 etc. does not need to define a single point in time but may rather refer to a (predefined) time range or time period, e.g. of 7, 10 or 20 days.

Therefore, the measurements of a test do not need to be taken at the same moment but at different moments within the time range. The method thus allows a certain temporal tolerance.

In a further embodiment, a computer program comprises computer-readable instructions which when executed by a data processing system cause the data processing system to carry out the method according to any one of methods described herein.

In a further embodiment, a recording medium readable by a computer and having recorded thereon a computer program includes instructions for executing the steps of a method according to any one of the methods described herein.

In a further embodiment, a processing device for determining a patient's response to a treatment in multiple myeloma, is configured to: obtain (or receive or provide) results of a series of predefined consecutive tests on the patient, determine a response at time t as a function of a test result of the time t and a subsequent test result of a time t+1.

In the present disclosure the terms “obtain”, “receive”, “provide” results may be used interchangeably, as the present disclosure is not limited on the way how the results are acquired, e.g. by taking physical measurements or by reading data from a data storage or by receiving a data from an external device, etc. It is merely desirable that the results are made available such that they can be processed by the method or the device according to the present disclosure. Also, the present disclosure is not limited to the timing of making the results available, e.g. providing all results simultaneously or only one by one (e.g. upon the measurement).

The foregoing general description and the following detailed description are exemplary and explanatory only and are not restrictive of the invention as claimed.

### BRIEF DESCRIPTION OF THE DRAWINGS

The foregoing summary as well as the following detailed description of preferred embodiments are better understood when read in conjunction with the appended drawings. For illustrating the invention, the drawings show exemplary details of systems, methods, and experimental data. The information shown in the drawings are exemplary and explanatory only and are not restrictive of the invention as claimed. In the drawings:

FIG. 1 shows a flowchart of a first example of a computer-implemented method of determining a patient's response to a treatment in multiple myeloma;

FIG. 2a shows a flowchart of a second example of a computer-implemented method of determining a patient's response to a treatment in multiple myeloma;

FIG. 2b shows a flowchart of a third example of a computer-implemented method of determining a patient's response to a treatment in multiple myeloma;

FIG. 3 shows schematic time diagrams of exemplary tests and determined responses;

FIGS. 4a and 4b show different exemplary roll-back functions used by the method of FIG. 1 or 2a or 2b; and

FIG. 5 shows a block diagram of a processing device for determining a patient's response to a treatment in multiple myeloma.

### DETAILED DESCRIPTION

The present disclosure relates to computer-implemented methods and systems for determining a patient's response to a treatment in multiple myeloma. The method may be implemented on a processing device.

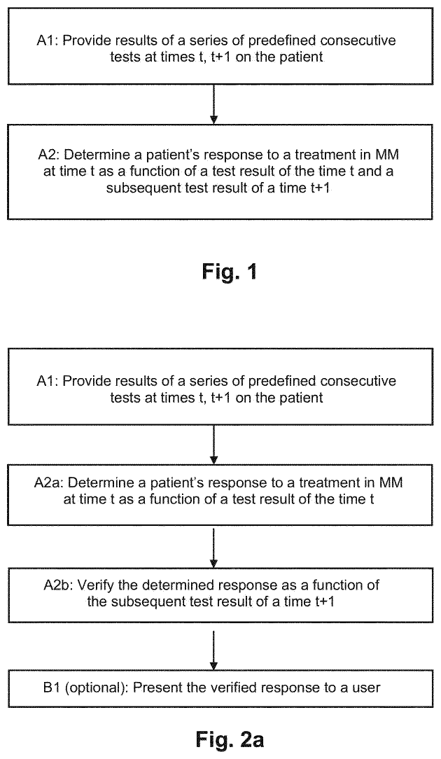

FIG. 1 shows a flowchart of a first example of a computer-implemented method of determining a patient's response to a treatment in multiple myeloma (MM).

In a step A1, results of a series of predefined consecutive tests at times t, t+1 (and optionally of further times t-n and/or t+n) on the patient are provided. For example, said results may be received from an external device or may be read from a data storage. It is also possible that the results are acquired by one or several sensors or medical devices and optionally be pre-processed to be provided to a processing device carrying out the method.

The consecutive test may hence be made at different times and may therefore be considered as consecutive tests. For example, a test may be made each month or every two or three months, e.g. after each treatment cycle. The times t and t+1, etc. may define a (tolerance) time range, e.g. of a predefined length, e.g. of 7 days. Therefore, the measurements of one test do not need to be taken at the same moment but at different moments within the time range.

Each test may comprise at least one predefined type of quantitative measurement on the patient or a combination of at least two predefined types of quantitative measurements on the patient. More in particular, each test may comprise the same type of measurement or types of measurements. In other words, the test may be repeated regularly, e.g. in predefined time intervals. Accordingly, based on a comparison of the test results (i.e. their respective measurement values) of different times, an evolution of the patient's state may be derived and the patient's treatment may be followed-up.

The results of the tests may be collected and be provided simultaneously (also cf. the second example). Alternatively, the results of the tests may be provided consecutively, once the results (i.e. the measurement values) have been obtained (also cf. the third example).

Generally, the tests, i.e. their determination of responses or their test measurements may correspond to the IMWG criteria but may not require all measurements defined in the IMWG criteria. The measurements used in the method of the present disclosure to determine the patient's response to a treatment in MM may therefore correspond to so-called ‘relaxed’ IMWG criteria. Therefore, the provision of measurements is less laborious and the determination of the response becomes possible in spite of the absence of measurements defined in the IMWG criteria. Examples of measurements are explained in more detail below.

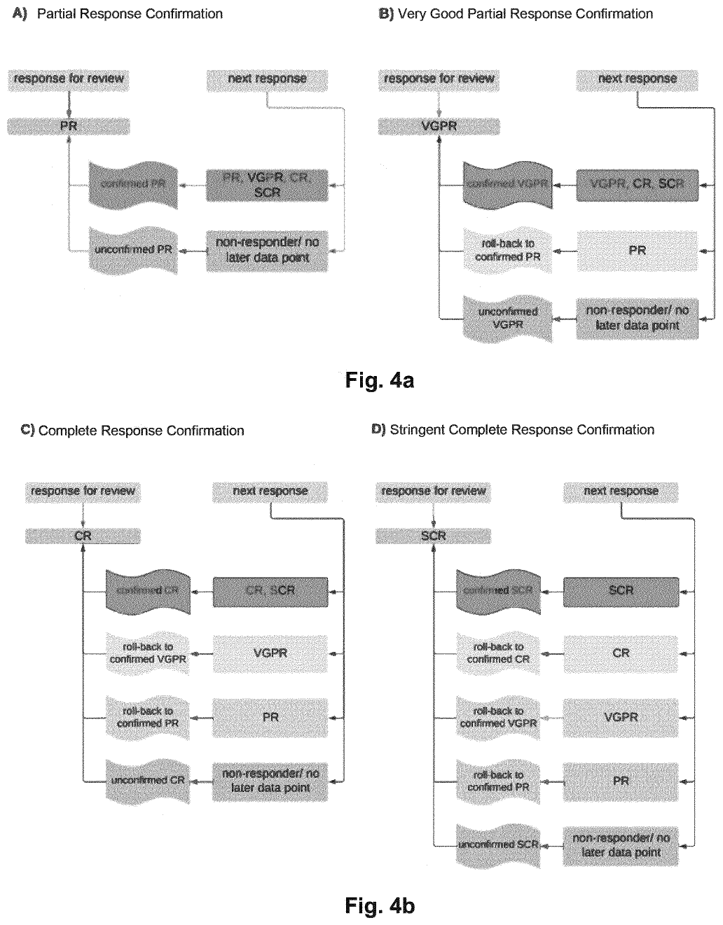

In a step A2, a patient's response to a treatment in MM at time t is determined as a function of a test result of the time t and a subsequent test result of a time t+1. The result of the subsequent test at time t+1 may thus be used to verify the result of the test at time t. For example, if the result of the subsequent test at time t+1 confirms the result of the test at time t, the test result at time t may be considered as valid and may be used to determine the response. In case the subsequent test result of time t+1 disagrees with (i.e. is worse than) the test result of time t, a so-called roll-back function may be applied to adapt the test result of time t, as explained in more detail below in context of FIGS. 4a and 4b.

The determined response may be presented to a user and/or may be provided to another method, algorithm or device for further processing.

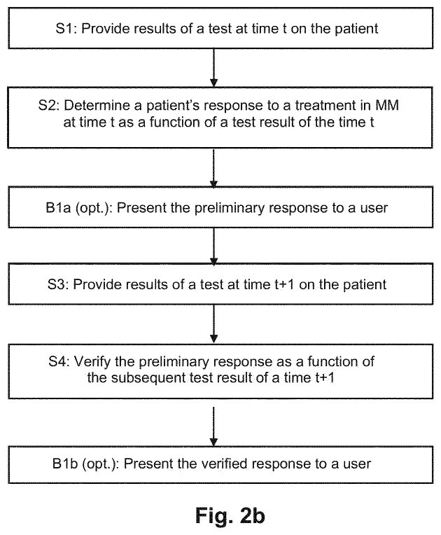

Steps A1 and A2 may be carried out simultaneously or subsequently, i.e. first step A1 and afterwards step A2. Exemplary embodiments of temporal aspects of the method are also described in context of FIGS. 2a and 2b.

By providing such a method, it becomes possible to increase the reliability of the determined patient's response. Therefore, the absence of measurements defined in the IMWG criteria can be compensated.

FIG. 2a shows a flowchart of a second example of a computer-implemented method of determining a patient's response to a treatment in multiple myeloma. The second example may generally correspond to the first example, i.e. comprise the same features, unless described differently. For example, the second example may be a more specific embodiment of the first example.

In a step A1, results of a series of predefined consecutive tests at times t, t+1 (and optionally of further times t-n and/or t+n) on the patient. Step A1 may correspond to step A1 of the first example. In particular, in the second example the results of the tests may be collected and be provided simultaneously.

In step A2a, a patient's response to a treatment in MM at time t is determined as a function of a test result of the time t.

In step A2b, the determined response is verified as a function of the subsequent test result of a time t+1. For example, if the result of the subsequent test at time t+1 confirms the result of the test at time t, the test result at time t may be considered as valid and may be used to determine the response. In case the subsequent test result of time t+1 disagrees with (i.e. is worse than) the test result of time t, a so-called roll-back function may be applied to adapt the test result of time t, as explained in more detail below in context of FIGS. 4a and 4b.

In an optional step B1, the verified response may be presented to a user, e.g. a user of the processing device. The user may for example be a physician. The verified response may for example be presented on an electronic display.

Accordingly, the presented verified response may be for example a confirmed response or an adapted response. In some cases, it may also be indicated to the user that the response cannot be confirmed, as explained in more detail below in context of FIGS. 4a and 4b.

FIG. 2b shows a flowchart of a third example of a computer-implemented method of determining a patient's response to a treatment in multiple myeloma;

The third example may generally correspond to the first example, i.e. comprise the same features, unless described differently. For example, the third example may be a more specific embodiment of the first example. The third example may be an alternative to the second example. In particular, in the third example the results of the tests may be provided consecutively, once the results (i.e. the measurement values) have been obtained (also cf. the third example).

In a step S1, results of a test at time t on the patient are provided. For example, this may happen, as soon as the respective measurements have been made and the test results (i.e. measurement values) are available.

In step S2, a patient's response to a treatment in MM at time t is determined as a function of a test result of the time t. Said determined response may also be referred to as a preliminary response, as it has not yet been confirmed by a subsequent test of time t+1.

In optional step B1a, the preliminary response is presented to a user.

Steps S1, S2 and/or B1a may be carried out simultaneously or subsequently, i.e. first step S1, afterwards step S2, and afterwards step B1a.

In step S3, results of a test at time t+1 on the patient are provided. For example, this may happen, as soon as the respective measurements have been made and the test results (i.e. measurement values) are available.

In step S4, the preliminary response is verified as a function of the subsequent test result of a time t+1.

For example, if the result of the subsequent test at time t+1 confirms the result of the test at time t, the preliminary response as valid. In case the subsequent test result of time t+1 disagrees with (i.e. is worse than) the test result of time t, a so-called roll-back function may be applied to adapt preliminary response, as explained in more detail below in context of FIGS. 4a and 4b.

In an optional step B1b, the verified response may be presented to a user. Said step may correspond to step B1 of the second example.

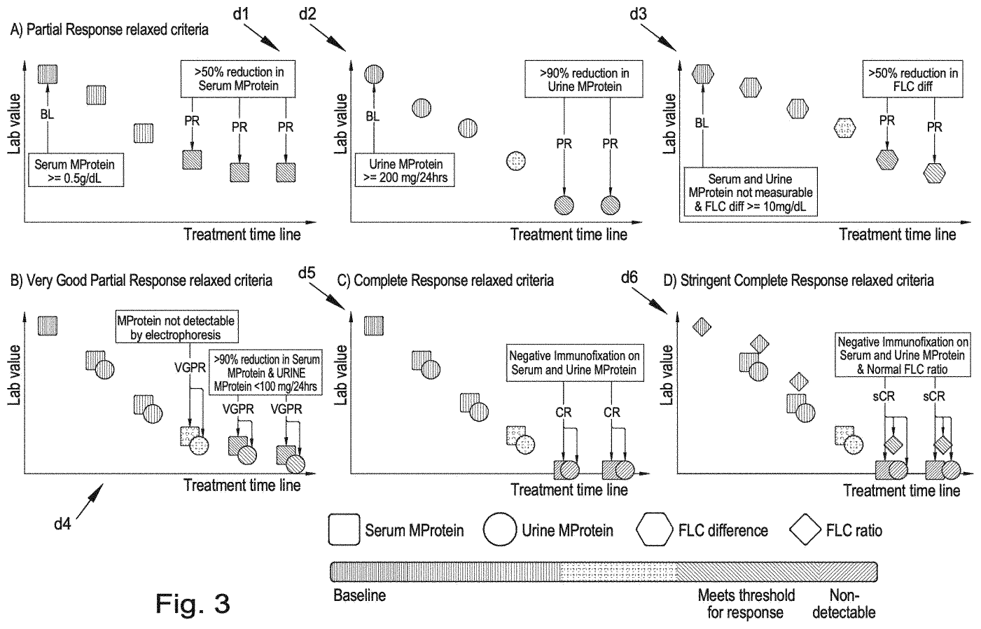

FIG. 3 shows schematic time diagrams of exemplary tests and determined responses.

In particular, FIG. 3 shows six time diagrams d1 to d6 which illustrate examples, of how patients may be tested (i.e. using different measurements) over time during the treatment. The examples further illustrate how the test results (i.e. measurement values or lab values) may evolve over time due to the treatment. Moreover, the patients' responses to the treatment, as determined based on the test results, are illustrated.

The determined response may comprise a plurality of increasing (e.g. qualitative) response levels. The plurality of increasing response levels may comprise:
a partial response (PR)
a very good partial response (VGPR),
a complete response (CR), and
a stringent complete response (SCR).

Conventionally, a physician or other qualified person would determine or estimate the response level at time t based on the test result at time t. However, according to the present disclosure the response level may be determined automatically using a predefined (computer-implemented) algorithm, as described in the following.

Generally, the method (i.e. the algorithm) uses test results of different times t, t+1 to determine a (confirmed) response level. Furthermore, the method may use tests comprising different types of measurements to determine the response level.

Moreover, the method does not require all IMWG criteria but merely needs to be in accordance with so-called ‘relaxed’ IMWG criteria:
In particular, measurement of data from bone marrow (BM) biopsies are excluded, what makes the method less laborious compared to a convention method due to the IMWG criteria.
Furthermore, a reduction in either serum or urine M protein levels (rather than both) may be required to determine a partial response (PR). When multiple values exist on the same date per patient, the largest value may be considered.
MCPROT in Urine and Serum values marked as “BELOW QUANTIFIABLE LIMITS” may be considered to be 0.
A window of [−7:+7] days may be implemented to allow matching of dates of labs at a time t (i.e., if a criterium requires both urine and serum M protein measurements, both measurements may merely be required to occur in the time window).
Depth of response may be confirmed by a second consecutive test (roll-back rules may be applied in absence of a confirmation, also cf. FIGS. 4a and 4b)
If multiple depths of response are experienced by a patient, the best response may be considered.
Immunofixation may be interpreted by: ONLY if the laboratory test value, LBORRES, is: LBORRES==‘NOT DETECTED’ (i.e. M protein is not detected), the result is considered negative, in case of missing data, the result is considered positive.

FIG. 3 shows in A) three diagrams d1 to d3 with exemplary lab values (i.e. exemplary evolutions) of different types of measurements according to relaxed IMWG criteria.

In diagram d1 the measurement type comprises measurements of the (e.g. monoclonal) myeloma (M) protein in the patient's serum, i.e. the serum M-protein. As schematically shown, the measured lab value of the serum M-protein decreases over time during the treatment. The example of diagram d1 shows six lab values taken over time (t, t+1, t+2 . . . ) which (at least in the beginning) are successively decreasing. In other words, the patient appears to respond to the treatment (at least in the beginning).

A baseline BL for starting a follow-up of the patient's response to the treatment may be determined, in case the measured serum M-protein is equal to or more than 0.5 g/dL (cf. first measurement).

Furthermore, a partial response (PR) may be determined, in case of a reduction of more than 50% of serum M-protein, (cf. the last three measurements).

In diagram d2 the measurement type comprises measurements of the (e.g. monoclonal) M-protein in the patient's urine, i.e. the urine M-protein.

A baseline BL for starting a follow-up of the patient's response to the treatment may be determined, in case the measured urine M-protein is equal to or more than 200 mg during 24 hours (cf. first measurement).

Furthermore, a partial response (PR) may be determined, in case of a reduction of urine M-protein by more than 90% in 24 hours or to less than 200 mg during 24 hours.

In diagram d3 the measurement type comprises measurements of the patient's free light chain (FLC) absolute difference, i.e. the FLC absolute difference.

A baseline BL for starting a follow-up of the patient's response to the treatment may be determined, in case the serum M-protein and urine M-protein are not measurable and the FLC absolute difference is equal to or more than 10 mg/dL (cf. first measurement).

Furthermore, a partial response (PR) may be determined, in case of a reduction of more than 50% in the FLC absolute difference.

Furthermore, the method may use tests comprising different types of measurements to determine the response level.

For example, FIG. 3 shows in B) a diagram d4 with exemplary lab values (i.e. an exemplary evolution) of different types of measurements according to relaxed IMWG criteria based on which a very good partial response may be determined.

The used measurement types comprise measurements of the serum M-protein and the urine M-protein. As schematically shown, the measured lab values decrease over time during the treatment. In other words, the patient appears to respond to the treatment (at least in the beginning). A very good partial response (VGPR) may be determined, once at least one or both the M-protein and the urine M-protein are not detectable any more by electrophoresis. In subsequent tests a very good partial response (VGPR) may be determined in case of a reduction of more than 90% in serum M-protein in combination with urine M-protein level being less than 100 mg in 24 hours.

Moreover, FIG. 3 shows in C) a diagram d5 with exemplary lab values (i.e. an exemplary evolution) of different types of measurements according to relaxed IMWG criteria based on which a complete response may be determined.

The used measurement types comprise measurement of the serum M-protein and the urine M-protein. The method of diagram d5 may thus correspond to that one of d4, however, the exemplary lab values of these diagrams may lead to different determined responses, i.e. VGPR in d4 and CR in d5.

In particular, as shown in the example of d5, a complete response (CR) may be determined, in case of a negative immunofixation on the serum and urine, more in particular in case the measured serum M protein becomes less than 1 g/dL and the measured urine M protein becomes less than 200 mg during 24 hours.

Moreover, FIG. 3 shows in D) a diagram d6 with exemplary lab values (i.e. an exemplary evolution) of different types of measurements according to relaxed IMWG criteria based on which a stringent complete response may be determined.

The used measurement types comprise measurements of the serum M-protein, the urine M-protein and the patient's free light chain (FLC) ratio, i.e. the FLC ratio. As schematically shown, the measured lab values decrease over time during the treatment. In other words, the patient appears to respond to the treatment (at least in the beginning). A stringent complete response (SCR) may be determined, in case of a negative immunofixation on the serum and urine and an FLC ratio being in a predefined normal range. Accordingly, the determination of a SCR may correspond to that one of a CR, wherein the SCR additionally requires measurement of the FLC ratio.

FIGS. 4a and 4b show different exemplary roll-back functions used by the method of FIG. 1 or 2a or 2b.

Generally, a depth of response (i.e. a response level determined based on a test at time t) may be confirmed by a second consecutive test at time t+1. If unconfirmed (i.e. in case the consecutive test at t+1 disagrees with the preceding test at time t), the response may be rolled-back (i.e. the determined response level may be decreased) to the assessed depth determined with the test at time t+1 or remains unconfirmed.

FIG. 4a shows in A) a roll-back function for a partial response (PR) confirmation, i.e. for the case that a test at time t leads to a PR.

The response for review (i.e. the preliminary response) may be determined by the test at time t. The next response may be determined by the test at time t+1.

In the example of A) the response for review may be a PR. In case the response level of the next response is at least as good as the that one of the response for review, i.e. a PR, VGPR, CR or SCR, the response for review may be confirmed. However, in case a next response cannot be determined or does not comprise any valid response level, the response for review is unconfirmed.

Furthermore, FIG. 4a shows in B) a roll-back function for a VGPR confirmation, i.e. for the case that a test at time t leads to a VGPR. The roll-back function of the example of B) may principally correspond to that one of the example of A). However, in B) the response for review may be a VGPR.

In case the response level of the next response is at least as good as the that one of the response for review, i.e. a VGPR, CR or SCR, the response for review may be confirmed. However, in case in case the response level of the next response is worse than that one of the response for review, i.e. only a PR, the response for review may be amended, i.e. rolled-back to a PR. Moreover, in case a next response cannot be determined or does not comprise any valid response level, the response for review is unconfirmed.

FIG. 4b shows in C) a roll-back function for a CR confirmation, i.e. for the case that a test at time t leads to a CR. The roll-back function of the example of C) may principally correspond to that one of the example of B). However, in C) the response for review may be a CR.

In case the response level of the next response is at least as good as the that one of the response for review, i.e. a CR or SCR, the response for review may be confirmed. However, in case in case the response level of the next response is worse than that one of the response for review, i.e. only a PR or VGPR, the response for review may be amended, i.e. rolled-back to a CR or VGPR, respectively. Moreover, in case a next response cannot be determined or does not comprise any valid response level, the response for review is unconfirmed.

Furthermore, FIG. 4b shows in D) a roll-back function fora SCR confirmation, i.e. for the case that a test at time t leads to a SCR. The roll-back function of the example of D) may principally correspond to that one of the example of C). However, in D) the response for review may be a SCR.

In case the response level of the next response is at least as good as the that one of the response for review, i.e. a SCR, the response for review may be confirmed. However, in case in case the response level of the next response is worse than that one of the response for review, i.e. only a PR, VGPR or CR, the response for review may be amended, i.e. rolled-back to a PR, VGPR or CR, respectively. Moreover, in case a next response cannot be determined or does not comprise any valid response level, the response for review is unconfirmed.

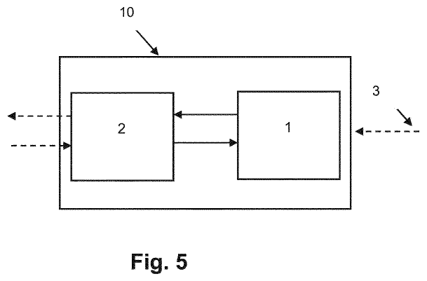

FIG. 5 shows a block diagram of a processing device 1 for determining a patient's response to a treatment in multiple myeloma and an optional display device 2.

The devices 1 and 2 may be comprised of an electronic device 10, for example a personal computer, a smartphone or a tablet computer. The processing device 1 and the optional display device 2 may however also be remote to each other. For example, the processing device 1 may be (or may part of or may comprise) a centralized server or part of a cloud application, and the display device 2 may be part of a client device, for example a smartphone, tablet computer or personal computer.

The processing device may comprise a processor, for example CPU and/or a GPU, and optionally a memory. More generally, the processing device may comprise digital circuits, computer-readable storage media, as one or more computer programs, or a combination of one or more of the foregoing. The computer-readable storage media may be non-transitory, e.g., as one or more instructions executable by a cloud computing platform and stored on a tangible storage device.

The processing device may receive the results of the tests, i.e. the measurement values 3 from an external device, e.g. from a data storage or via a data interface from another system comprising e.g. one or several sensors. The display device 2 may display the determined response.

The method may be implemented as a computer program comprising computer-readable instructions which when executed by processing device 1 cause the processing device 1 to carry out the method according to the present disclosure. However, the method according to the present disclosure may also be carried out by an external processing device (not shown in FIG. 5), i.e. remote to the electronic device 10.

In an experimental test, the method according to the present disclosure was based on ‘relaxed’ IMWG criteria, which entail exclusion of bone marrow biopsies data and of imaging results, and reduction in either serum or urine M protein levels (rather than both) to assign partial response. This method was applied to patient-level clinical trial data from the Bellini trial (Kumar et al. Lancet Oncol 2020) with response assignment made by an independent review committee (IRC) in the trial used as the ‘ground truth’, cf. e.g.: Kumar S K, Harrison S J, Cavo M, et al. Venetoclax or placebo in combination with bortezomib and dexamethasone in patients with relapsed or refractory multiple myeloma (BELLINI): a randomised, double-blind, multicentre, phase 3 trial. Lancet Oncol. 2020 December; 21(12):1630-1642 . . . . Agreement between the IRC's and method's assignments of response was estimated using Cohen's Kappa statistic.

Differences in overall response rate (ORR) between treatment and placebo arms were calculated using stratified Cochran-Mantel Haenszel tests based on strata at randomization with number of prior lines of therapy and previous proteasome inhibitor treatment status variables used for stratification.

The experimental test lead to the following results: The Bellini trial is a Phase 3 clinical trial with 2:1 design enrolling 194 and 97 patients in its treatment and placebo arms, respectively (291 patients in total). Regular assessments of M protein and FLC levels were performed (median 13 or 14 measurements), providing detailed trajectory of patient response.

Hereinbelow, ‘algorithm’ refers to the algorithm based on ‘relaxed’ IMWG criteria according to the present disclosure. A comparison between the IRC's and algorithm's assignments of responders classified as Partial Response or better (PR+) resulted in almost perfect agreement with Cohen's Kappa 0.83 (275/291 assignments in agreement). The Cohen's Kappa between the IRC's and Investigator's assessments is 0.85, suggesting an upper limit to the attainable performance of the algorithm's performance and indicating that the algorithm's error is within some expected uncertainty. Due to the exclusion of BM information, agreement decreased to 0.56 when depths of response were considered separately as opposed to grouping PR+ patients together with most cases being overestimated as complete response (CR) or stringent CR (SCR).

In assessing the treatment effect (i.e. the patients' response), differences in ORR between the intervention and placebo arms in the trial based on IRC's assessment could be accurately recapitulated (ORR in intervention vs placebo arms derived from IRC response assessment in the Bellini trial was: 82% [159/194] vs 68% [66/97], Odds Ratio (OR) 2.10, indicating the benefits of the treatment. The ORR derived from the algorithm according to the present disclosure was: 87% [168/194] vs 73% [71/97], Odds Ratio 2.31). Based on these results, algorithm response assignment led to consistent conclusions about treatment efficacy in the Bellini trial.

Implementation of criteria to characterize very good partial response or better (VGPR+) also led to a conclusion consistent with the Bellini trial IRC's assessment. Characterization of very good partial response or better (VGPR+) by the algorithm was consistent with the IRC's assessment (VGPR+ in intervention vs. placebo arms by IRC: 59% [114/194] vs. 36% [35/97], OR 2.48; by the algorithm: 60% [116/194] vs. 37% [36/97], OR 2.49).

Characterization of criteria of CR+ resulted in estimates of treatment efficacy that were lower, but directionally in agreement (CR+ in intervention vs. placebo arms by IRC: 26% [51/194] vs. 5% [5/97], OR 6.47; by the algorithm: 39% [76/194] vs. 23% [22/97], OR 2.19). This discrepancy can again be linked to the ‘relaxed’ IMWG criteria and the over assignment of CR/SCR in the absence of BM evaluation.

As a consequence, the method according to the present disclosure allows using ‘relaxed’ IMWG criteria to account for routine clinical practice settings and demonstrates very high agreement with the assessment of trained clinicians, while reliably reproducing the efficacy analysis in the Bellini trial when ORR and VGPR+ are considered.

To confirm the algorithm's ability to reliably assess a response to a MM treatment even for incomplete electronic health records, results of a sensitivity analysis are discussed in the following: Missing data were simulated by randomly excluding 50% of all laboratory measurements. The levels of data reduction were chosen to reflect the availability of laboratory measurements previously observed in real-world data.

The original laboratory data contained two consecutive measurements of the same type for 284/291 patients (data missing for seven patients who discontinued the trial early) and thus confirmed response criteria were met for these patients. Upon reduction of 50% or 75% of the laboratory measurements, 278/291 and 277/291 patients 227 were eligible for confirmed response criteria evaluation, respectively, with the remainder (13/291 and 14/291, respectively) automatically assigned as non-responders. Randomly removing 50% of all laboratory measurements per patient resulted in a minor reduction in the algorithm's accuracy in assessing the number of patients with PR+, with 265/291 assignments in agreement with the IRC (Cohen's Kappa 0.75). The number of responders assigned by the algorithm decreased when missing data were introduced, as a second laboratory measurement that would have confirmed response was lost in some patients, thus leading to misclassification. Concordance decreased (Cohen's Kappa 0.48) when depths of response were considered separately.

As a consequence, the method and algorithm according to the present disclosure shows that use ‘relaxed’ IMWG criteria provides very high agreement with the assessment of trained clinicians, while reliably reproducing the efficacy analysis in the Bellini trial even in the presence of incomplete health data records.

In this specification the phrase “configured to” is used in different contexts related to computer systems, hardware, or part of a computer program. When a system is said to be configured to perform one or more operations, this means that the system has appropriate software, firmware, and/or hardware installed on the system that, when in operation, causes the system to perform the one or more operations. When some hardware is said to be configured to perform one or more operations, this means that the hardware includes one or more circuits that, when in operation, receive input and generate output according to the input and corresponding to the one or more operations. When a computer program is said to be configured to perform one or more operations, this means that the computer program includes one or more program instructions, that when executed by one or more computers, causes the one or more computers to perform the one or more operations.

Unless otherwise stated, the foregoing alternative examples are not mutually exclusive, but may be implemented in various combinations to achieve unique advantages. In the foregoing description, the provision of the examples described, as well as clauses phrased as “such as,” “including” and the like, should not be interpreted as limiting embodiments to the specific examples; rather, the examples are intended to illustrate only one of many possible embodiments.

Further implementations are summarized in the following examples:

Example 1: A computer-implemented method of determining a patient's response to a treatment in multiple myeloma, comprising: providing results of a series of predefined consecutive tests on the patient, determining a response at time t as a function of a test result of the time t and a subsequent test result of a time t+1.

Example 2. The method according to example 1, wherein each test comprises at least one predefined type of quantitative measurement of the patient or a combination of at least two predefined types of quantitative measurements of the patient.

Example 3. The method according to example 1 or 2, wherein the response is a qualitative response value and/or the response comprises a plurality of increasing qualitative response levels.

Example 4. The method according to the preceding examples, wherein the plurality of increasing qualitative response levels comprise: a partial response (PR), a very good partial response (VGPR), a complete response (CR), and a stringent complete response (SCR).

Example 5. The method according to any one of the preceding examples, wherein the response and/or the response level at time t is determined as a function of the test result of time t using a predefined algorithm, and/or the subsequent test result of time t+1 is used to confirm the determined response and/or determined response level at time t.

Example 6. The method according to any one of the preceding examples, wherein in case the subsequent test result of time t+1 is worse than the test result of time t, the determined response level at time t is decreased as a function of the subsequent test result and/or is decreased to a response level corresponding to the subsequent test result.

Example 7. The method according to any one of the preceding examples, wherein the different types of measurements comprise at least one of: measurement of the Myeloma (M) protein in the patient's serum, i.e. the serum M-protein, measurement of the (M) protein in the patient's urine, i.e. the urine M-protein, measurement of the patient's free light chain (FLC) absolute difference, i.e. the FLC absolute difference, and measurement of the patient's free light chain (FLC) ratio, i.e. the FLC ratio, and/or the tests exclude a bone marrow biopsy of the patient, and/or the response is determined without test results of a bone marrow biopsy of the patient.

Example 8. The method according to any one of the preceding examples 4 to 7, wherein a partial response (PR) and/or a very good partial response (VGPR) is determined as a function of the measured serum M-protein or the measured urine M-protein, and optionally of the FLC absolute difference.

Example 9. The method according to any one of the preceding examples 4 to 8, wherein a partial response (PR) is determined as a function of: a reduction of more than 50% of serum M-protein, or a reduction of urine M-protein by more than 90% in 24 hours or to less than 200 mg in 24 hours, or, if the serum and urine M-protein are unmeasurable, a more than 50% decrease in the difference between involved and uninvolved FLC levels.

Example 10. The method according to any one of the preceding examples 4 to 9, wherein a very good partial response (VGPR) is determined as a function of: a detectability of serum and urine M-protein by immunofixation but not on electrophoresis, or a reduction of more than 90% in serum M-protein in combination with urine M-protein level being less than 100 mg in 24 hours.

Example 11. The method according to any one of the preceding examples 4 to 10, wherein a complete response (CR) and/or stringent complete response (SCR) is determined as a function of the measured serum M protein being less than 1 g/dL and the measured urine M protein being less than 200 mg during 24 hours.

Example 12. The method according to any one of the preceding examples 4 to 11, wherein a complete response (CR) is determined as a function of: a negative immunofixation on the serum and urine, and/or a stringent complete response (SCR) is determined as a function of: a negative immunofixation on the serum and urine, and an FLC ratio being in a predefined normal range.

Example 13. The method according to any one of the preceding examples, wherein at least one of the times t and t+1, or each of the times t, t+1, t+n of the series defines a time range.

Example 14. A computer program comprising computer-readable instructions which when executed by a data processing system cause the data processing system to carry out the method according to any one of preceding examples.

Example 15. A processing device for determining a patient's response to a treatment in multiple myeloma, configured to: obtain results of a series of predefined consecutive tests on the patient, determine a response at time t as a function of a test result of the time t and a subsequent test result of a time t+1.

1. A computer-implemented method of determining a patient's response to a treatment in multiple myeloma, comprising:
providing results of a series of predefined consecutive tests on the patient, and
determining a response at time t as a function of a test result of the time t and a subsequent test result of a time t+1.

2. The method according to claim 1, wherein each test comprises at least one predefined type of quantitative measurement of the patient or a combination of at least two predefined types of quantitative measurements of the patient.

3. The method according to claim 1, wherein the response is a qualitative response value and/or the response comprises a plurality of increasing qualitative response levels.

4. The method according to claim 3, wherein the plurality of increasing qualitative response levels comprise:
a partial response, PR,
a very good partial response, VGPR,
a complete response, CR, and
a stringent complete response, SCR.

5. The method according claim 1, wherein
the response and/or the response level at time t is determined as a function of the test result of time t using a predefined algorithm, and/or
the subsequent test result of time t+1 is used to confirm the determined response and/or determined response level at time t.

6. The method according to claim 1, wherein in case the subsequent test result of time t+1 is worse than the test result of time t, the determined response level at time t is decreased as a function of the subsequent test result and/or is decreased to a response level corresponding to the subsequent test result.

7. The method according to claim 1, wherein the different types of measurements comprise at least one of:
measurement of the Myeloma, M-protein in the patient's serum, i.e. the serum M-protein,
measurement of the M-protein in the patient's urine, i.e. the urine M-protein,
measurement of the patient's free light chain, FLC, absolute difference, i.e. the FLC absolute difference, and
measurement of the patient's free light chain, FLC, ratio, and/or
the tests exclude a bone marrow biopsy of the patient, and/or
the response is determined without test results of a bone marrow biopsy of the patient.

8. The method according to claim 4, wherein a partial response, PR, and/or a very good partial response, VGPR, is determined as a function of the measured serum M-protein or the measured urine M-protein, and optionally of the FLC absolute difference.

9. The method according to claim 4, wherein a partial response, PR, is determined as a function of:
a reduction of more than 50% of serum M-protein, or
a reduction of urine M-protein by more than 90% in 24 hours or to less than 200 mg in 24 hours, or
if the serum and urine M-protein are unmeasurable, a more than 50% decrease in the difference between involved and uninvolved FLC levels.

10. The method according to claim 4, wherein a very good partial response, VGPR, is determined as a function of:
a detectability of serum and urine M-protein by immunofixation but not on electrophoresis, or
a reduction of more than 90% in serum M-protein in combination with urine M-protein level being less than 100 mg in 24 hours.

11. The method according to claim 4, wherein a complete response, CR, and/or stringent complete response, SCR, is determined as a function of the measured serum M protein being less than 1 g/dL and the measured urine M protein being less than 200 mg during 24 hours.

12. The method according to claim 4, wherein a complete response, CR, is determined as a function of:
a negative immunofixation on the serum and urine, and/or
a stringent complete response, SCR, is determined as a function of:
a negative immunofixation on the serum and urine, and
an FLC ratio being in a predefined normal range.

13. The method according to claim 1, wherein at least one of the times t and t+1, or each of the times t, t+1, . . . , t+n of the series defines a time range.

14. A computer program comprising computer-readable instructions which when executed by a data processing system cause the data processing system to carry out the method of claim 1.

15. A processing device for determining a patient's response to a treatment in multiple myeloma, configured to:
obtain results of a series of predefined consecutive tests on the patient, and
determine a response at time t as a function of a test result of the time t and a subsequent test result of a time t+1.
